(19) United States
(12) Patent Application Publication
(10) Pub. No.: US 2023/0126287 A1
(43) Pub. Date: Apr. 27, 2023

(54) METHODS AND SYSTEMS FOR DISINFECTING SURGICAL SITE

(71) Applicant: GYRUS ACMI, INC. D/B/A OLYMPUS SURGICAL TECHNOLOGIES AMERICA, Westborough, MA (US)

(72) Inventors: Jordan N. Milford, Bethlehem, PA (US); Joey Magno, Dudley, MA (US); Kester Julian Batchelor, Mound, MN (US); Charles A. Baker, Rogers, MN (US)

(21) Appl. No.: 18/047,093

(22) Filed: Oct. 17, 2022

Related U.S. Application Data
(60) Provisional application No. 63/262,852, filed on Oct. 21, 2021.
(60) Provisional application No. 63/269,066, filed on Mar. 9, 2022.

(51) Int. Cl. A61K 33/18; A61N 1/44; A61N 5/06; A61M 31/00; A61K 33/20; A61K 35/74

(52) U.S. Cl. CPC A61K 33/18; A61N 1/44; A61N 5/0624; A61M 31/00; A61N 5/0601; A61K 33/20; A61K 35/74; A61N 2005/0661; A61M 2205/053

(57) ABSTRACT

A method of performing a surgical procedure comprises inserting a medical instrument into anatomy of a patient to reach target tissue within an anatomical cavity, disinfecting anatomy within the patient with the medical instrument and performing treatment of the target tissue with the medical instrument. A medical device for providing disinfecting to a surgical site, the medical device comprises an elongate shaft extending from a proximal end to a distal end, an imaging device located proximate the distal end of the elongate shaft, a lighting element located proximate the distal end of the elongate shaft, a working channel extending at least partially through the elongate shaft and a disinfecting system attached to the elongate shaft to deliver a disinfecting capability proximate the distal end of the elongate shaft.

### PRIORITY CLAIM

This application claims the benefit of priority to U.S. Provisional Application No. 63/262,852, filed on Oct. 21, 2021, and U.S. Provisional Application No. 63/269,066, filed on Mar. 9, 2022, the contents of which are incorporated herein by reference in their entireties.

### TECHNICAL FIELD

This document pertains generally, but not by way of limitation, to surgical systems and methods for preparing an anatomic site for surgery. More specifically, but not by way of limitation, the present application relates to systems and methods for disinfecting a surgical site to prevent infection.

### BACKGROUND

Many surgical procedures involve the treatment or removal of target tissue, e.g., diseased, potentially diseased or otherwise unwanted tissue, located inside of a patient. As such, some of these procedures require access to the internal anatomy of the patient via an open procedure or through a smaller opening in minimally invasive (e.g., laparoscopic) procedures. In some endoscopy cases, the patient anatomy is accessed through the mouth or anus, as well as any natural orifice as can be used in urology, gynecology, ear-nose-throat (ENT) procedures, without producing an opening or incision in the patient to reach an internal cavity or duct within the patient, such as the gastrointestinal (GI) tract. These endoscopy procedures can be referred to as endolumenal procedures because the procedures take place inside a tube, duct or hollow organ in the body. Some endolumenal procedures involve the removal of tissue from a tissue wall forming the duct or cavity. As such, it is typical for such procedures to be performed without the use of antiseptic since the internal cavity or duct is not intended to be punctured and the endoscopy instruments do not enter other sterile portions of the anatomy. For example, bacteria of the gastrointestinal duct typically does not enter into other portions of the body outside the gastrointestinal system for endolumenal procedure performed in the stomach.

### OVERVIEW

The present inventors have recognized, among other things, that problems to be solved in performing endolumenal surgical procedures include the growing potential for unintended punctures or penetrations through a duct wall where the non-sterile region of the surgery is undesirably put into communication with the sterile region of the body. As mentioned, one major difference between endolumenal surgery and open or laparoscopic surgery is in preparation of the surgical site. In a laparoscopic or open procedures, povidone-iodine (PVP-I) is routinely used as an antiseptic for skin disinfection before and after surgery. The PVP-I is typically applied to the skin around the area of incision before starting the procedure. Endolumenal procedures like Polypectomy, Endoscopic Mucosal Resection (EMR), or Endoscopic Submucosal Dissection (ESD) are used to remove tissue within the Gastro-Intestinal (GI) tract. Typically, endolumenal procedures are designed to remove the upper layers of the GI tract and therefore not reach any sterile cavities within the body. However, one complication associated with many GI procedures is perforation of a GI tract wall, which allows for the microbes within the GI tract to potential access a sterile cavity in the body. In addition, more invasive procedures are being developed as the market shifts towards more endolumenal techniques and many of these procedures intentionally access a sterile cavity, including, for example, Full-Thickness Resection (FTR) and endoscopic ultra-sound (EUS) drainage using a stent. Since the GI tract inherently has significant microbial activity, this could lead to post-procedure infections and complications.

The present subject matter can provide solutions to this problem and other problems, such as by providing medical devices and systems, such as catheters, endoscopes, robots and other delivery devices, that can be configured and employed to deliver disinfectant to a surgical site. The disinfectant can be applied intra-operatively inside the lumen of the endolumenal procedure to prevent or inhibit the spread of bacteria or other contaminants through the lumen wall, due to intended or unintended perforation. The disinfectant can also be applied such that the spread of bacteria or other contaminants between anatomic ducts or lumens can be prevented

The present subject matter can provide solutions to this problem and other problems, such as by providing systems and methods involving applying an antibacterial or antiseptic formula with the assistance of a catheter or other device or instrument within an anatomic lumen at the site where an endolumenal procedure is to be performed. The disinfecting catheter can be inserted through the working channel of a scope or a robot, alongside the scope or robot, or can be integrated into a scope or a robot. In examples, forward water jet functionality of the scope or robot can be used to spray antiseptic. A delivery device can also be used as or with an accessory or auxiliary delivery system. Examples of accessory or auxiliary delivery systems include a pump, compressed gas, a syringe, etc.

In examples, the present disclosure can utilize a spray catheter or hollow catheter delivered through the working channel of an endoscope, laparoscope, or robot. The spray catheter or hollow catheter can be used to spray a liquid, gel, or powder in the lumen or in the laparoscopy space to destroy microbial proteins and/or DNA. In additional examples, ultra-violet light and cold plasma can be used as a disinfecting media, in addition to liquids and powders.

In an example, a method of performing a surgical procedure can comprise inserting a medical instrument into anatomy of a patient to reach target tissue within an anatomical cavity, disinfecting anatomy within the patient with the medical instrument, and performing treatment of the target tissue with the medical instrument.

In another example, a medical device for providing disinfecting to a surgical site, the medical device can comprise an elongate shaft extending from a proximal end to a distal end, an imaging device located proximate the distal end of the elongate shaft, a lighting element located proximate the distal end of the elongate shaft, a working channel extending at least partially through the elongate shaft, and a disinfecting system attached to the elongate shaft to deliver a disinfecting capability proximate the distal end of the elongate shaft.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

FIG. 1 is a schematic diagram of an endoscopy system comprising an imaging and control system and an endoscope with which the disinfecting systems, devices and methods of the present disclosure can be used.

FIG. 2 is a schematic diagram of the imaging and control system of FIG. 1 showing the imaging and control system and disinfecting system connected to the endoscope.

FIG. 3A is a schematic top view of a camera module including optical components for a side-viewing endoscope and an elevator mechanism.

FIG. 3B is an enlarged cross-sectional view taken along the plane 3B-3B of FIG. 3A showing the optical components.

FIG. 3C is an enlarged cross-sectional view taken along the plane 3C-3C of FIG. 3A showing the elevator mechanism.

FIG. 4A is an end view of a camera module including optical and functional components suitable for use with the endoscope of FIGS. 1 and 2.

FIG. 4B is a cross-sectional view taken along the plane 4B-4B of FIG. 4A showing components of the camera module.

FIG. 5A is a schematic view of an endoscope connected to a disinfecting system including a disinfectant reservoir and a spray adjusting device.

FIG. 5B is perspective view of an example of an endoscope having an integrated disinfecting system of the present disclosure.

FIG. 5C is an end view of the endoscope of FIG. 5B showing various passages therethrough.

FIG. 5D is a schematic illustration of an endolumenal surgical site where an instrument is protruding from an esophageal duct to enter an internal cavity of the stomach where a tumor is located, the instrument being used to distribute disinfectant onto the tumor.

FIG. 5E-5G illustrate steps of a endoscopic submucosal dissection (ESD) procedure that can be performed in conjunction with using one of more of the disinfecting systems and methods described herein.

FIG. 6A is perspective view of an example of an endoscope having an external disinfecting system of the present disclosure.

FIG. 6B is an end view of the endoscope of FIG. 6A showing various passages therethrough.

FIG. 7A is perspective view of an example of an endoscope having a radial disinfecting system of the present disclosure comprising ultraviolet lighting.

FIG. 7B is a cross-sectional view of the endoscope of FIG. 7A showing a lighting ring attached to a light conductor.

FIG. 8 is end view of an example of an endoscope having a radial disinfecting system of the present disclosure comprising a disinfecting coating.

FIG. 9 is a line diagram illustrating methods for disinfecting surgical sites according to the present disclosure.

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

### DETAILED DESCRIPTION

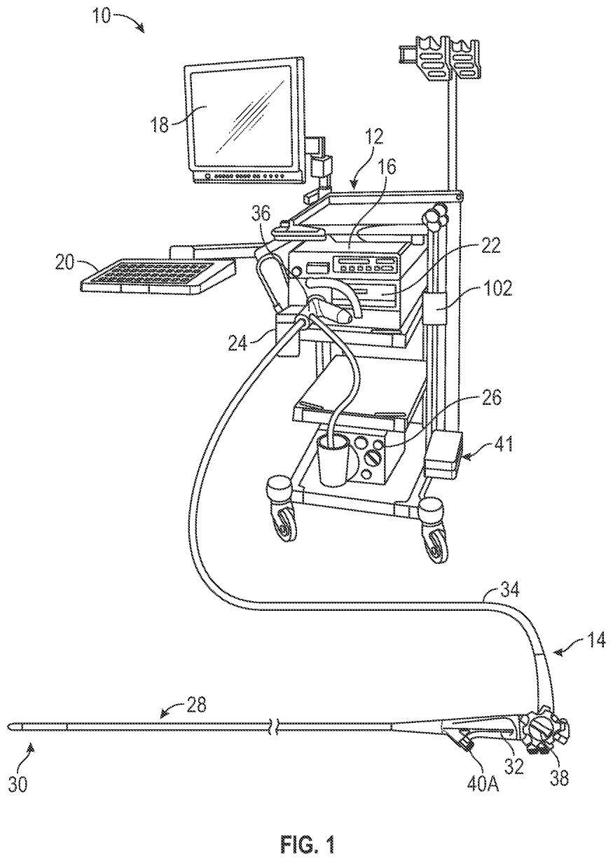

FIG. 1 is a schematic diagram of endoscopy system 10 comprising imaging and control system 12 and endoscope 14. The system of FIG. 1 is an illustrative example of an endoscopy system suitable for use with the systems, devices and methods described herein, such as those including the capability of delivering or applying disinfecting and sanitizing capabilities. According to some examples, endoscope 14 can be insertable into an anatomical region for imaging and/or to provide passage of or attachment to one or more surgical instruments, such as forceps and the like, for treatment of a disease state associated with the anatomical region. Endoscope 14 can, in advantageous aspects, interface with and connect to imaging and control system 12. In the illustrated example, endoscope 14 comprises a scope suitable for performing endoluminal procedures, though other types of endoscopes, including duodenoscopes, cystoscopes, ureteroscopes, renoscopes, nephroscopes, and cholangioscopes, can be used with the features and teachings of the present disclosure.

Imaging and control system 12 can comprise control unit 16, output unit 18, input unit 20, light source 22, fluid source 24, suction pump 26 and disinfecting system 102.

Imaging and control system 12 can include various ports for coupling with endoscopy system 10. For example, control unit 16 can include a data input/output port for receiving data from and communicating data to endoscope 14. Light source 22 can include an output port for transmitting light to endoscope 14, such as via a fiber optic link. Fluid source 24 can include a port for transmitting fluid to endoscope 14. Fluid source 24 can comprise a pump and a tank of fluid or can be connected to an external tank, vessel or storage unit. Suction pump 26 can comprise a port used to draw a vacuum from endoscope 14 to generate suction, such as for withdrawing fluid from the anatomical region into which endoscope 14 is inserted. Output unit 18 and input unit 20 can be used by an operator of endoscopy system 10 to control functions of endoscopy system 10 and view output of endoscope 14. Control unit 16 can additionally be used to generate signals or other outputs from treating the anatomical region into which endoscope 14 is inserted. In examples, control unit 16 can generate electrical output, acoustic output, a fluid output, a gas output, and the like for treating the anatomical region with, for example, cauterizing, cutting, freezing and the like. Control unit 16 can be used to operate disinfecting system 102 and various instruments, components and sub-systems thereof.

Endoscope 14 can comprise insertion section 28, functional section 30 and handle section 32, which can be coupled to cable section 34 and coupler section 36. Coupler section 36 can be connected to control unit 16 to connect to endoscope 14 to multiple features of control unit 16, such as input unit 20, light source unit 22, fluid source 24 and suction pump 26.

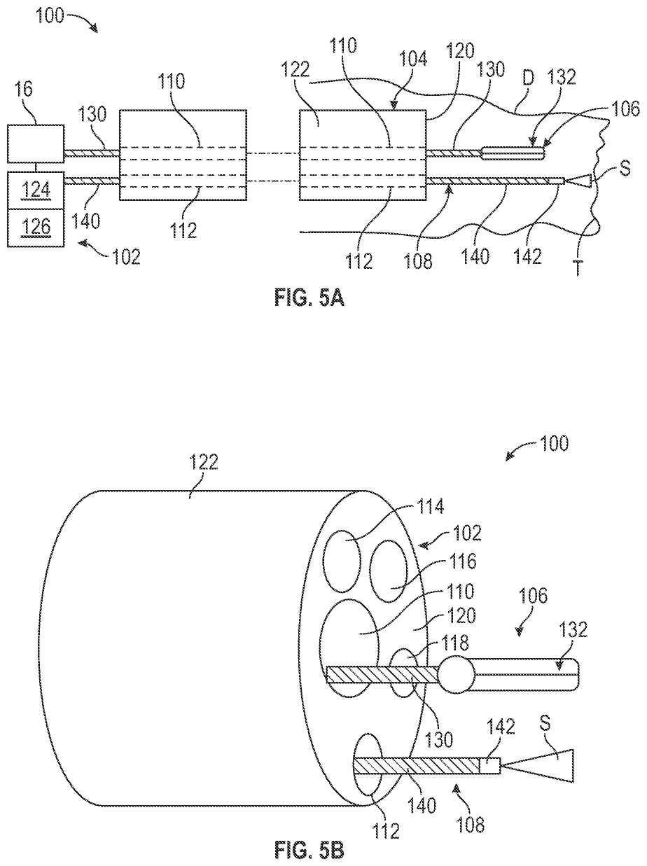

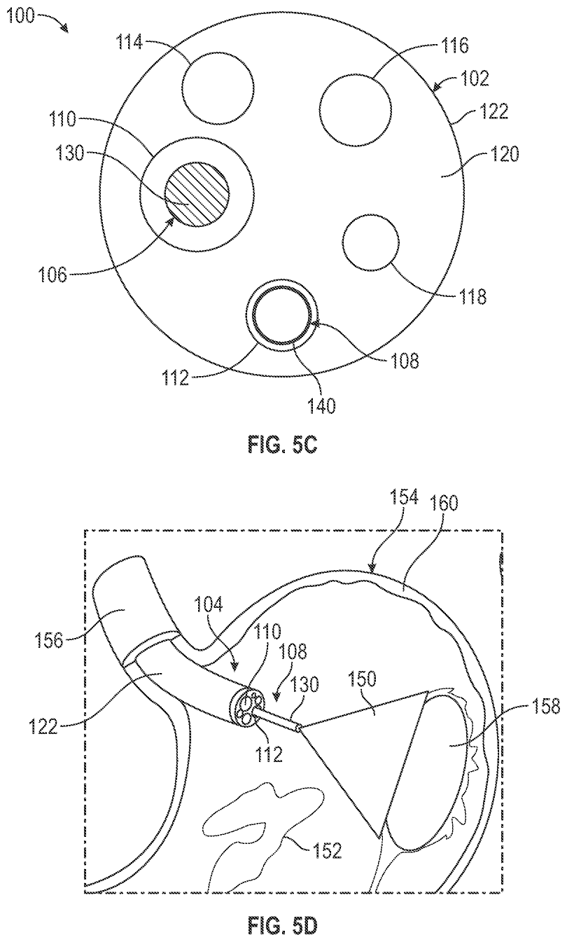

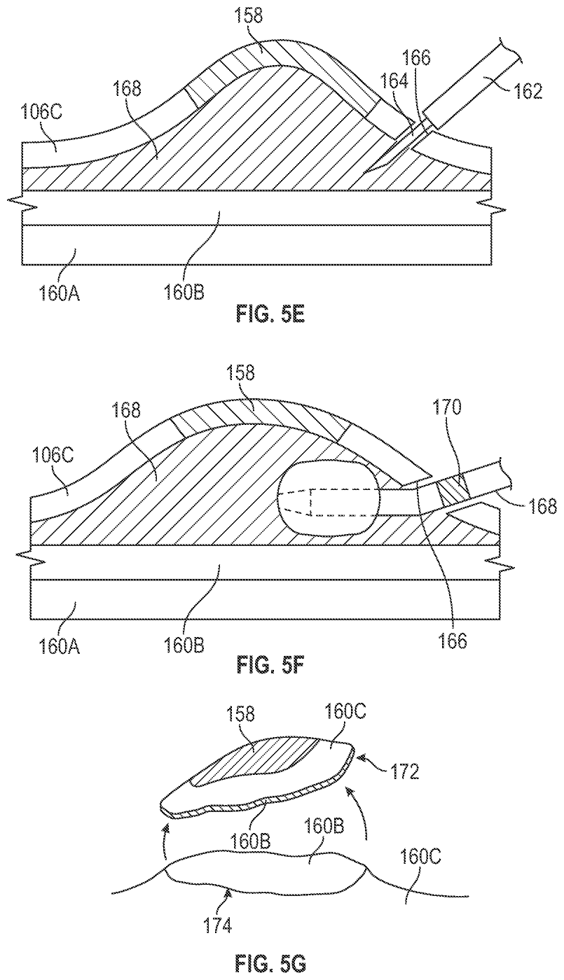

Insertion section 28 can extend distally from handle section 32 and cable section 34 can extend proximally from handle section 32. Insertion section 28 can be elongate and include a bending section, and a distal end to which functional section 30 can be attached. The bending section can be controllable (e.g., by control knob 38 on handle section 32) to maneuver the distal end through tortuous anatomical passageways (e.g., stomach, duodenum, kidney, ureter, etc.). Insertion section 28 can also include one or more working channels (e.g., an internal lumen) that can be elongate and support insertion of one or more therapeutic tools of functional section 30, such as endoscopy system 100 of FIGS. 5A and 5B. The working channel can extend between handle section 32 and functional section 30. Additional functionalities, such as fluid passages, guide wires, and pull wires can also be provided by insertion section 28 (e.g., via suction or irrigation passageways, and the like).

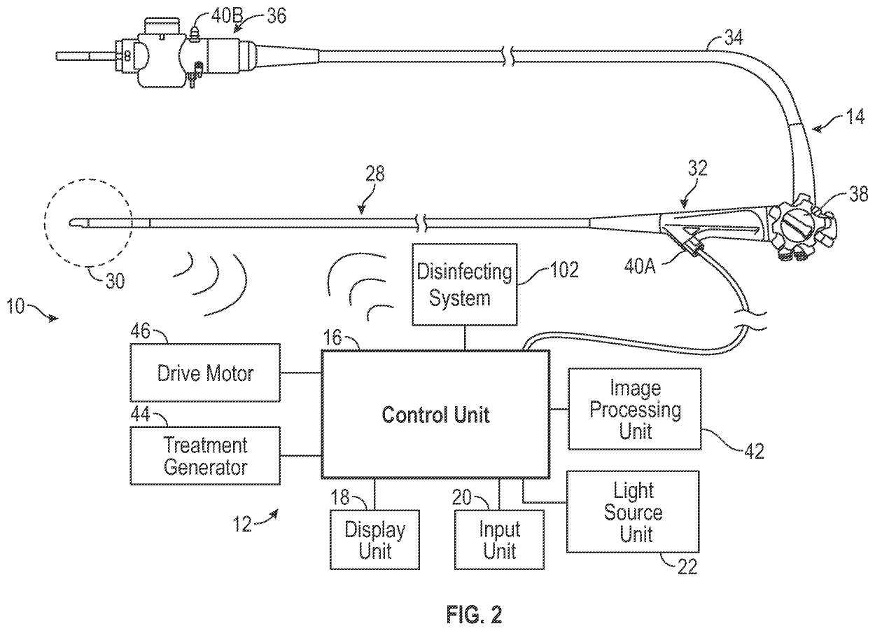

Handle section 32 can comprise knob 38 as well as port 40A. Knob 38 can be coupled to a pull wire, or other actuation mechanisms, extending through insertion section 28. Port 40A, as well as other ports, such as port 40B (FIG. 2), can be configured to couple various electrical cables, guide wires, auxiliary scopes, tissue collection devices, fluid tubes and the like to handle section 32 for coupling with insertion section 28. For example, tissue separator device 106 can be fed into endoscope 14 via port 40A.

Imaging and control system 12, according to examples, can be provided on a mobile platform (e.g., cart 41) with shelves for housing light source 22, suction pump 26, image processing unit 42 (FIG. 2), etc. Alternatively, several components of imaging and control system 12 shown in FIGS. 1 and 2 can be provided directly on endoscope 14 so as to make the endoscope “self-contained.”

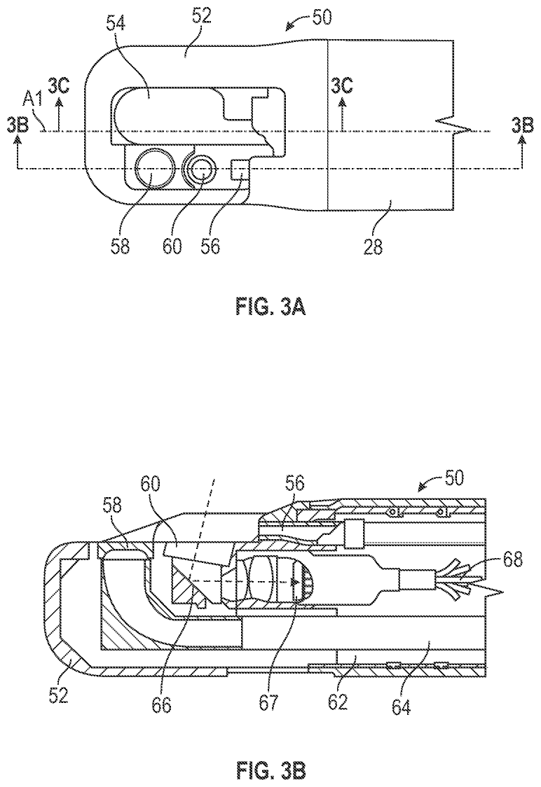

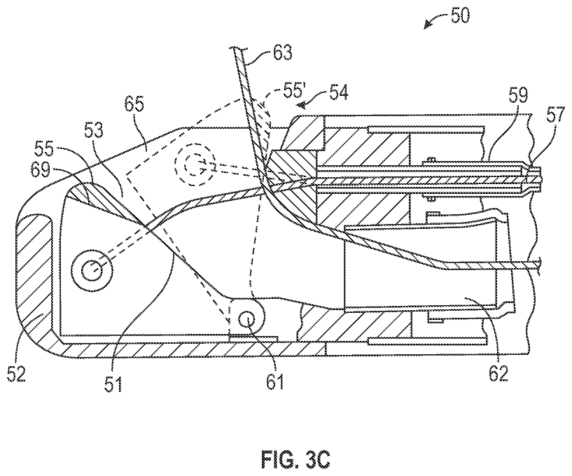

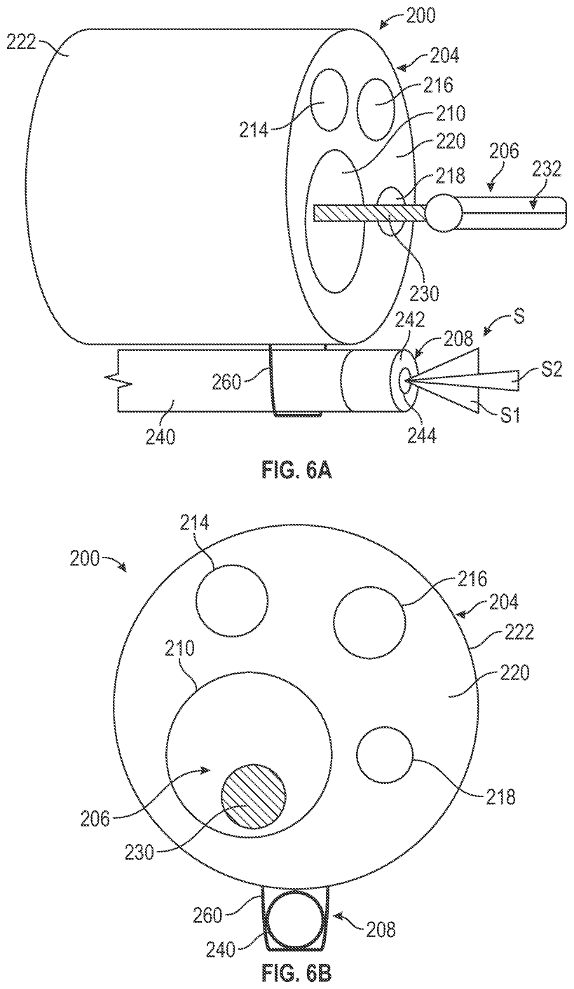

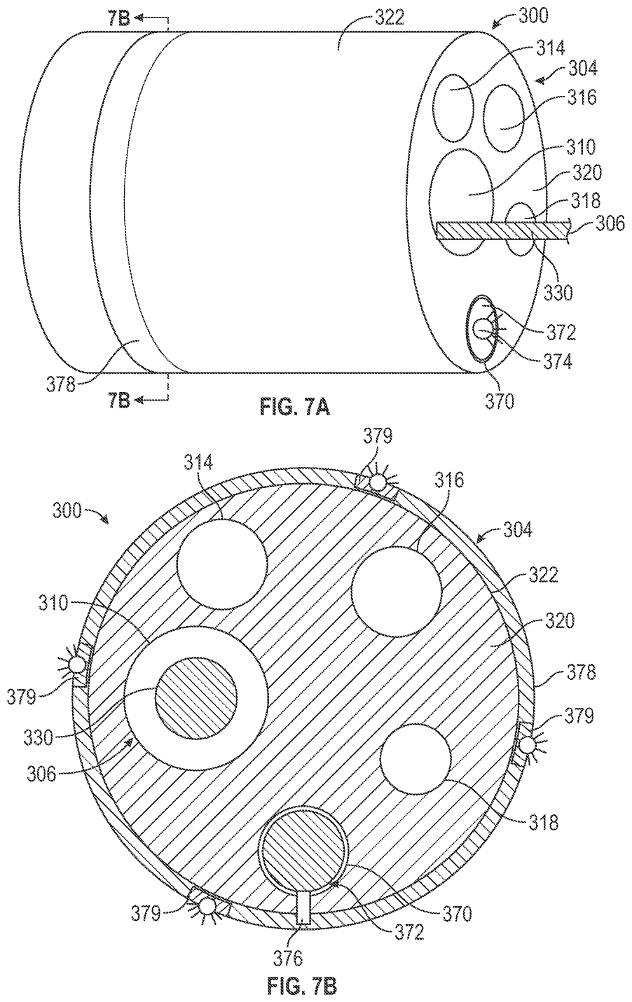

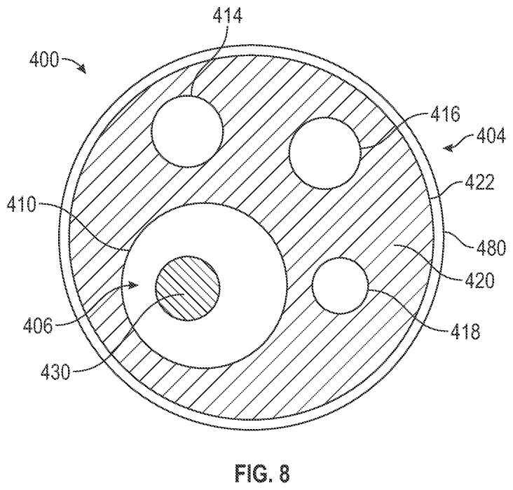

Functional section 30 can comprise components for treating and diagnosing anatomy of a patient. Functional section 30 can comprise an imaging device, an illumination device and an elevator, such as is described further with reference to elevator 54 of FIGS. 3A-3C. Functional section 30 can further comprise disinfectant applying systems. For example, functional section 30 can comprise one or more passages for applying liquid or powder disinfectant (e.g., FIGS. 5A and 5B), ultraviolet light (e.g., FIGS. 7A and 7B) and cold plasma (e.g., FIGS. 5A and 5B). In other examples, functional section 30 can be coupled to an external disinfectant applying systems (e.g., FIGS. 6A and 6B).

FIG. 2 is a schematic diagram of endoscopy system 10 of FIG. 1 comprising imaging and control system 12 and endoscope 14. FIG. 2 schematically illustrates components of imaging and control system 12 coupled to endoscope 14, which in the illustrated example comprises a duodenoscope. Imaging and control system 12 can comprise control unit 16, which can include or be coupled to image processing unit 42, treatment generator 44 and drive unit 46, as well as light source 22, input unit 20 and output unit 18. Coupler section 36 can be connected to control unit 16 to connect to endoscope 14 to multiple features of control unit 16, such as image processing unit 42 and treatment generator 44. In examples, port 40A can be used to insert another instrument or device, such as a daughter scope or auxiliary scope, into endoscope 14. Such instruments and devices can be independently connected to control unit 16 via cable 47. In examples, port 40B can be used to connect coupler section 36 to various inputs and outputs, such as video, air, light and electric. As is discussed below in greater detail with reference to FIGS. 5A-5C, control unit 16 can comprise, or can be in communication with, disinfecting system 102. Disinfecting system 102 can comprise various components for applying various forms of disinfectant, such as liquid reservoirs, powder reservoirs, gas tanks, pumps, compressors, cold plasma generators, ultra-violet light generators and the like. Control unit 16 can be configured to activate a camera to view target tissue distal of endoscope 14. Likewise, control unit 16 can be configured to activate light source unit 22 to shine light on surgical instruments extending from endoscope 14.

Image processing unit 42 and light source 22 can each interface with endoscope 14 (e.g., at functional unit 30) by wired or wireless electrical connections. Imaging and control system 12 can accordingly illuminate an anatomical region, collect signals representing the anatomical region, process signals representing the anatomical region, and display images representing the anatomical region on display unit 18. Imaging and control system 12 can include light source 22 to illuminate the anatomical region using light of desired spectrum (e.g., broadband white light, narrow-band imaging using preferred electromagnetic wavelengths, and the like). Imaging and control system 12 can connect (e.g., via an endoscope connector) to endoscope 14 for signal transmission (e.g., light output from light source, video signals from imaging system in the distal end, diagnostic and sensor signals from a diagnostic device, and the like).

Fluid source 24 (FIG. 1) can be in communication with control unit 16 and can comprise one or more sources of air, saline or other fluids, as well as associated fluid pathways (e.g., air channels, irrigation channels, suction channels) and connectors (barb fittings, fluid seals, valves and the like). Fluid source 24 can be utilized as a system for delivering disinfectant as part of the present disclosure, such as by operating as a disinfectant delivery system or by operating as an irrigation delivery system that can be selectively converted to operating as a disinfectant delivery system intra-operatively. Fluid source 24 can operate in conjunction with disinfecting system 102. Imaging and control system 12 can also include drive unit 46, which can be an optional component. Drive unit 46 can comprise a motorized drive for advancing a distal section of endoscope 14, as described in at least PCT Pub. No. WO 2011/140118 A1 to Frassica et al., titled “Rotate-to-Advance Catheterization System,” which is hereby incorporated in its entirety by this reference.

FIGS. 3A-3C illustrate a first example of functional section 30 of endoscope 14 of FIG. 2. FIG. 3A illustrates a top view of functional section 30 and FIG. 3B illustrates a cross-sectional view of functional section 30 taken along section plane 3B-3B of FIG. 3A. FIGS. 3A and 3B each illustrate “side-viewing endoscope” (e.g., duodenoscope) camera module 50. In side-viewing endoscope camera module 50, illumination and imaging systems are positioned such that the viewing angle of the imaging system corresponds to a target anatomy lateral to central longitudinal axis A1 of endoscope 14.

In the example of FIGS. 3A and 3B, side-viewing endoscope camera module 50 can comprise housing 52, elevator 54, fluid outlet 56, illumination lens 58 and objective lens 60. Housing 52 can form a fluid tight coupling with insertion section 28. Housing 52 can comprise opening for elevator 54. Elevator 54 can comprise a mechanism for moving a device inserted through insertion section 28. In particular, elevator 54 can comprise a device that can bend an elongate device extended through insertion section 28 along axis A1, as is discussed in greater detail with reference to FIG. 3C. Elevator 54 can be used to bend the elongate device at an angle to axis A1 to thereby treat the anatomical region adjacent side-viewing endoscope camera module 50. Elevator 54 is located alongside, e.g., radially outward of axis A1, illumination lens 58 and objective lens 60.

As can be seen in FIG. 3B, insertion section 28 can comprise central lumen 62 through which various components can be extended to connect functional section 30 with handle section 32 (FIG. 2). For example, illumination lens 58 can be connected to light transmitter 64, which can comprise a fiber optic cable or cable bundle extending to light source 22 (FIG. 1). Likewise, objective lens 60 can be coupled to prism 66 and imaging unit 67, which can be coupled to wiring 68. Also, fluid outlet 56 can be coupled to fluid line 69, which can comprise a tube extending to fluid source 24 (FIG. 1). Other elongate elements, e.g., tubes, wires, cables, can extend through lumen 62 to connect functional section 30 with components of endoscopy system 10, such as suction pump 26 (FIG. 1) and treatment generator 44 (FIG. 2).

FIG. 3C a schematic cross-sectional view taken along section plane 3C-3C of FIG. 3A showing an elevator 54. Elevator 54 can comprise deflector 55 that can be disposed in space 53 of housing 52. Deflector 55 can be connected to wire 57, which can extend through tube 59 to connect to handle section 32. Wire 57 can be actuated, such as by rotating a knob, pulling a lever, or pushing a button on handle section 32. Movement of wire 57 can cause rotation, e.g., clockwise, from a first position of deflector 55 about pin 61 to a second position of deflector 55, indicated by 55′. Deflector 55 can be actuated by wire 57 to move the distal portion of instrument 63 extending through window 65 in housing 52.

Housing 52 can comprise accommodation space 53 that houses deflector 55. Instrument 63 can comprise forceps, a catheter, or the like that extends through lumen 62. Instrument 63 can also comprise a disinfecting device described herein or another device, such as an endoscope that can receive a disinfecting device described herein. A proximal end of deflector 55 can be attached to housing 62 at pin 61 8 provided to the rigid tip 21. A distal end of deflector 55 can be located below window 65 within housing 62 when deflector 55 is in the lowered, or un-actuated, state. The distal end of deflector 55 can at least partially extend out of window 65 when deflector 55 is raised, or actuated, by wire 57. Instrument 63 can slide on angled ramp surface 51 of deflector 55 to initially deflect the distal end of instrument 63 toward window 65. Angled ramp surface 51 can facilitate extension of the distal portion of instrument 63 extending from window 65 at a first angle relative to the axis of lumen 62. Angled ramp surface 51 can include groove 69, e.g. a v-notch, to receive and guide instrument 63. Deflector 55 can be actuated to bend instrument 63 at a second angle relative to the axis of lumen 62, which is closer to perpendicular that the first angle. When wire 57 is released, deflector 55 can be rotated, e.g., counter-clockwise, back to the lowered position, either by pushing or relaxing of wire 57.

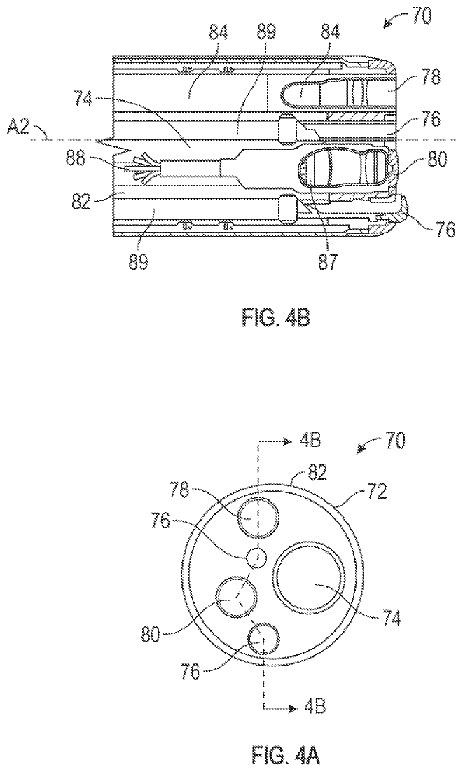

FIGS. 4A and 4B illustrate an example of functional section 30 of endoscope 14 of FIGS. 1 and 2. FIG. 4A illustrates an end view of functional section 30 and FIG. 4B illustrates a cross-sectional view of functional section 30 taken along section plane 5B-5B of FIG. 5A. FIGS. 4A and 4B each illustrate “end-viewing endoscope” (e.g., gastroscope, colonoscope, cholangioscope, etc.) camera module 70. In end-viewing endoscope camera module 70, illumination and imaging systems are positioned such that the viewing angle of the imaging system corresponds to a target anatomy located adjacent an end of endoscope 14 and in line with central longitudinal axis A2 of endoscope 14. Camera module 70 can be incorporated into any of the devices described herein, such as those shown in FIGS. 5A-8.

In the example of FIGS. 4A and 4B, end-viewing endoscope camera module 70 can comprise housing 72, therapy unit 74, fluid outlets 76, illumination lens 78 and objective lens 80. Housing 72 can comprise and endcap for insertion section 28, thereby providing a seal to lumen 82.

As can be seen in FIG. 4B, insertion section 28 can comprise lumen 82 through which various components can be extended to connect functional section 30 with handle section 32 (FIG. 2). For example, illumination lens 78 can be connected to light transmitter 84, which can comprise a fiber optic cable or cable bundle extending to light source unit 22 (FIG. 2). Likewise, objective lens 80 can be coupled to imaging unit 87, which can be coupled to wiring 88. Also, fluid outlets 76 can be coupled to fluid lines 89, which can comprise a tube extending to fluid source 24 (FIG. 2). In examples, one of fluid outlets 76 can comprise an inlet connected to a fluid line 89 configured for suction, such as being connected to a vacuum, for recovery of lavage and irrigation fluid. Other elongate elements, e.g., tubes, wires, cables, can extend through lumen 82 to connect functional section 30 with components of endoscopy system 10, such as suction pump 26 (FIG. 2) and treatment generator 44 (FIG. 2). For example, therapy unit 74 can comprise a wide-diameter lumen for receiving other treatment components, such as cutting devices and therapeutic devices including medical instrument 106.

Endoscope camera module 70 can also include a photosensitive element, such as a charge-coupled device (“CCD” sensor) or a complementary metal-oxide semiconductor (“CMOS”) sensor. In either example, imaging unit 87 can be coupled (e.g., via wired or wireless connections) to image processing unit 42 (FIG. 2) to transmit signals from the photosensitive element representing images (e.g., video signals) to image processing unit 42, in turn to be displayed on a display such as output unit 18. In various examples, imaging and control system 12 and imaging unit 87 can be configured to provide outputs at desired resolution (e.g., at least 480p, at least 720p, at least 1080p, at least 4K UHD, etc.) suitable for endoscopy procedures.

As discussed in greater detail below, endoscope 14 can comprise systems and devices for providing disinfectant, e.g., liquids, powders, cold plasma and ultra-violet light, to anatomy of a patient either directly or with the use of another instrument or device. For examples, FIGS. 5A and 5B show a disinfectant delivery system incorporated into a shaft of a scope and FIGS. 6A and 6B show a disinfectant delivery system attached to the outside of a shaft of a scope.

FIG. 5A is a schematic view of endoscopy system 100 connected to disinfecting system 102. Endoscopy system 100 can comprise endoscope 104, medical instrument 106 and disinfectant instrument 108. Endoscope 104 can be inserted into anatomic duct D to reach target tissue T. Endoscope 104 can be inserted into endoscope 14 of FIGS. 1 and 2 to reach target tissue T.

FIG. 5B is perspective view of endoscopy system 100 comprising disinfecting system 102, endoscope 104, medical instrument 106 and disinfectant instrument 108. FIG. 5C is a distal end view of endoscopy system 100 of FIG. 5B showing working channel 110, disinfectant channel 112, objective lens 114, illumination lens 116, and fluid outlet 118 in end face 120 of shaft 122. Disinfecting system 102 can comprise motive device 124 and disinfecting substance reservoir 126. FIGS. 5A-5C are discussed concurrently.

Medical instrument 106 can be inserted into working tool channel 112 to obtain tissue from the patient. Disinfectant instrument 108 can be inserted into disinfectant channel 112 to deliver a disinfecting material to anatomy of a patient. Disinfectant instrument 108 and medical instrument 106 can be inserted into and withdrawn from endoscope 104 before or after insertion of endoscope 104 from duct D.

Objective lens 114 can be configured similarly as objective lens 80 of FIGS. 4A and 4B. Objective lens 114 can be configured to direct light toward an imaging unit to provide digital images to output unit 18. Illumination lens 116 can be configured similarly as illumination lens 78 of FIG. 4A and 4B. Illumination lens 116 can be configured to direct light from a light transmitter, such as a light transmitter that receives light from light source unit 22, toward tissue distal of distal end face 120, thereby illuminating tissue for medical instrument 106 and disinfecting instrument 108. Fluid outlet 118 can be configured similarly as fluid outlets 76 of FIGS. 4A and 4B. One or more fluid outlets 118 can be provided to deliver and recover fluids, such as by being coupled to a fluid source or a suction source. Specifically, fluid outlet 118 can be connected to fluid source 24 and suction pump 26 (FIG. 1). In examples, fluid outlet 118 can be used to deliver disinfectant, such as by providing fluid source 24 with disinfectant. In examples, a sanitizing packet can be added to fluid source 24 at a desired point in time during a procedure to provide sanitizing capabilities. The sanitizing packet can be manually added to fluid source 24 or can be added to fluid source 24 by an automatic dispensing mechanism of control unit 16 (FIG. 1).

Elongate shaft 122 of endoscope 104 can additionally be provided with steering capabilities as is described with reference to scope 14. For example, elongate shaft 122 can include pull wires that can be coupled to an actuation device to impart curvature to elongate shaft 122.

Working channel 110 and disinfectant channel 112 can be configured to receive a working tool, such as medical instrument 106, and a disinfecting system, such as disinfecting instrument 108, respectively. Working channel 110 and disinfectant channel 112 can extend from distal end face 120 to a proximal portion of elongate shaft 122. For examples, proximal ends of channel 110 and disinfectant channel 112 can be coupled to a port, such as port 40 of FIG. 1, configured to allow a working tool to enter elongate shaft 122. The cross-sectional area or diameter of working channel 110 and disinfectant channel 112 can be sized to allow for medical instrument 106 to pass freely therethrough.

Medical instrument 106 can comprise a tissue retrieval device such as a forceps or any other device suitable for separating, retrieving or collecting sample biological matter. Medical instrument 106 can comprise shaft 130 and tissue separators 132. Shaft 130 of medical instrument 106 can comprise a pliable body that can allow tissue separator 132 to be angled out of elongate shaft 122. Shaft 130 can additionally accommodate passage of control features, such as actuation wires, to tissue separator 132 to facilitate actuation of tissue separator 132 to collect tissue.

Disinfecting instrument 108 can be configured to deliver a disinfecting substance, such as a gas, liquid or powder, stored in substance reservoir 126. Disinfecting instrument 108 can comprise elongate body 140, such as a tube or hose having a lumen through which disinfecting substance can flow or be dispensed. In examples, elongate body 140 can be open at a distal end to allow disinfecting substance to flow freely therefrom. In example, disinfecting instrument 108 can comprise dispenser 142. Dispenser 142 can comprise a device for controlling or shaping flow of disinfecting substance from elongate body 140, such as a nozzle and the like. Examples of dispenser 142 are discussed in greater detail with reference to FIG. 6A. In additional examples, elongate body 140 can comprise conductor wires and gas tubes extending between disinfecting system 102 and dispenser 142, and dispenser 142 can comprise an electrode for a cold plasma applicator. A proximal end portion of disinfecting instrument 108 can be connected to one or all of disinfecting system 102, fluid source 24 and control unit 16 (FIG. 1). Motive device 124 can be operated via a user input to obtain disinfecting substance from reservoir 126 and provide disinfecting substance to dispenser 142. In examples, motive device 124 can comprise a fluid pump, a compressor and the like. Elongate body 140 can be steered or curved via operation of elongate shaft 122 of endoscope 104 to direct disinfecting substance S onto target tissue T. Thus, a user of system 100 can operate disinfecting system 102 and endoscope 104 to selectively dispense a disinfecting substance, media or agent to internal anatomic areas during a procedure.

The systems and methods of the present disclosure can utilize various substances and compositions as the disinfecting substance or as an antiseptic. A variety of formulations are possible and could be in the form of a liquid, gel, or powder. Attributes of the disinfecting substance or antiseptic can comprise:

1. Destroy microbial proteins and/or DNA quickly;
2. Biocompatible with little or no side effects for the patient;
3. Antiseptic should stay in the area sprayed and provide some visual difference between treated and untreated areas;
4. Visual difference dissipates, does not affect visualization during the procedure, or enhances the visualization during the remainder of the procedure; and
5. Reasonable cost in mass production.

Suitable formulations for the disinfectant can comprise Povidone-iodine (PVP-I), which can be ingested in limited quantities, and Hypochlorous acid (HOCl), which has disinfection properties and naturally occurs within the body.

In examples, disinfecting instrument 108 can be omitted and disinfecting substance can be provided by disinfecting system 102 directly to disinfecting channel 112. In such examples, disinfecting channel 112 can comprise a leak-proof passage such as a tube or conduit that can convey liquid or powder disinfecting material from reservoir 126 to dispenser 142.

In examples, disinfecting instrument 108 can be provided on the exterior of shaft 122 and disinfecting channel 112 can be omitted, as discussed with reference to FIGS. 6A and 6B.

In examples, disinfecting instrument 108 can be configured to operate with a plasma system. For example, motive device 124 and reservoir 126 can be replaced with or comprise components and devices for producing a cold plasma jet or plume at dispenser 142. In examples, motive device 124 and reservoir 126 can be replaced with or comprise one or more gas sources, one or more electrical generators, one or more gas tubes, one or more conductors and one or more electrodes. The gas sources can include supplies of gases suitable for the formation of cold plasma, such as oxygen, helium, argon, nitrogen, heliox and air. In examples, a carrier gas from a gas source, such as at reservoir 126, can be passed through a gas tube extending through shaft 140, conducting wires extending along the gas tube can be connected to electrodes on opposite sides of a discharge end of the gas tube, an electric generator can provide current through the conducting wires to produce a dielectric barrier between the electrodes, and the dielectric barrier can ignite the carrier gas into cold plasma. In examples, cold plasma can be produced by disinfecting system 102 via dielectric barrier discharge methods, floating electrode dielectric barrier discharge methods, radio frequency plasma jets, pulsed direct current-driven plasma jets and the like, as is described in Hoffmann C, Berganza C, Zhang J. Cold Atmospheric Plasma: methods of production and application in dentistry and oncology. Med Gas Res. 2013; 3(1): 21. Published 2013 Oct. 1. doi:10.1186/2045-9912-3-21, the contents of which are hereby incorporated into the present application by this reference.

In examples, versions of disinfecting instrument 108 configured for delivering disinfecting plasma are described in U.S. Pat. No. 11,197,708 to Cheng, titled “Plasma generator configured for use with an auxiliary device,” which is assigned to Gyms ACMI, Inc., the contents of which are incorporated herein by this reference.

FIG. 5D is a schematic diagram illustrating a method of delivering disinfectant 150 to an internal anatomic location using a disinfectant instrument 108. Endoscope 104 can be inserted into cavity 152 of organ 154 via duct 156. In examples, organ 154 can comprise a stomach such that cavity 152 comprises the interior of the stomach and duct 156 can comprise the esophagus. Endoscope 104 can be configured to disinfect the esophagus while being delivered to the stomach, such as by including radial-oriented disinfecting light capabilities, such as UV-C light emitters, as is discussed below in greater detail with reference to FIGS. 7A and 7B, or by including a coating capable of emitting a disinfecting substance, as discussed below in greater detail with reference to FIG. 8. Endoscope 104 can include disinfectant channel 112 into which disinfectant instrument 108 can be inserted. As mentioned, disinfectant instrument 108 can additionally be inserted through working channel 110 of endoscope 104 or attached to the outside of endoscope 104. Endoscope 104 can be operated to point disinfectant instrument 108 toward target tissue 158 on duct wall 160. As illustrated, dispenser 142 can be configured to spray disinfectant 150 in a triangular or conical pattern. However, dispenser 142 can be controlled by a user to change or vary the shape of the spray pattern to, for example, widen or narrow the triangular or conical pattern to provide disinfectant 150 on larger or smaller areas, respectively, as is described with reference to FIG. 6A.

Disinfectant instrument 108 can be extended from disinfectant channel 112 to reach the location of target tissue 158. Endoscope 104 can be steered to guide disinfectant instrument 108 toward target tissue 158. Imaging capabilities of endoscope 104 can be used to guide disinfectant instrument 108. As mentioned, disinfectant instrument 108 can comprise a catheter or separate instrument extending from endoscope 104. However, disinfectant instrument 108 can be integrated into endoscope 104 such that disinfectant 150 can be emitted directly from endoscope 104. Additionally, disinfectant instrument 108 can be provided alongside endoscope 104. Disinfectant instrument 108 can be selectively operated by a surgeon or user to spray, dispense, emit or otherwise release disinfectant 150 in the form of gas, liquid, gel, powder, plasma, light or other forms.

Examples of procedures that can be performed using the systems, devices and methods of the present disclosure include Endoscopic Submucosal Dissection (ESD) procedures. Exemplary ESD procedures are described in U.S. Pat. No. 9,402,683 to Yamano et al., titled “Submucosal layer dissection instrument, submucosal layer dissection system, and submucosal layer dissection method,” which is assigned to Olympus Corporation, the contents of which are incorporated herein by this reference. A brief description of an ESD procedure is provided as an example procedure where the disinfectant methods and systems of the present disclosure can be used. Although, other types of procedures can be used with the methods and systems of the present disclosure.

As shown in FIG. 5E, target tissue 158 can be located on duct wall 160. An ESD procedure attempts to remove only upper layers of a duct wall where the target tissue is to be removed. Duct wall 160 can be made up of various layers, including muscular layer 160A, submucosal layer 160B and mucosal layer 160C. Target tissue 158 can be located only in mucosal layer 160C or in mucosal layer 160C and submucosal layer 160B. It is desirable to leave the muscularis and serosa layers intact, though all or some of the muscularis layer can be removed. As such, it can be desirable to only remove a portion of mucosal layer 160C that includes target tissue 158, along with some or none of submucosal layer 160B, while leaving muscular layer 160A intact to prevent biological matter from within cavity 152 from passing into other anatomic portions of the body on the opposite side of muscular layer 160A and mucosal layer 160C. For example, it is desirable to avoid the spread of bacteria and digestive matter from within the stomach, e.g., organ 154, to outside the gastrointestinal tract or other sterile portions of the body outside of the stomach, e.g., organ 154. Likewise, it is desirable to prevent the spread of fecal matter in the intestines to outside the gastrointestinal tract. In order to guard against potential spread of bacteria, target tissue 158, e.g., the tumor, as well as surrounding tissue can be treated with a disinfectant, as discussed herein.

In a first step of the ESD procedure, the boundary of target tissue 158 can be marked using a surgical instrument. In examples, a cauterizing instrument can be used to mark indicia in mucosal layer 160C around the perimeter of target tissue 158. The boundary can be marked approximately 5 mm to 10 mm away from edges of target tissue 158. For example, a surgical instrument can comprise electrodes that can be activated by an energy source, such as at control unit 16 (FIG. 1), to cauterize portions of mucosal layer 160C. The marked boundary can be useful in visualizing the boundary of target tissue 158 in subsequent steps where tissue can become stretched or distorted.

In a second step of the ESD procedure, as shown in FIG. 5E, mucosal layer 160C can be separated from muscular layer 160A. In examples, surgical instrument 162 can be inserted through endoscope 104 (FIG. 5D) to engage duct wall 160. Surgical instrument 162 can comprise needle 164 that can puncture duct wall 160 at aperture 166 to deliver saline. In other examples, a glycerin or a gas can be introduced into duct wall 160. The saline, glycerin or gas can cause submucosal layer 160B to separate from mucosal layer 160C, thereby causing a distancing of mucosal layer 160C from muscular layer 160A. The indica marked around the perimeter of target tissue 158 in the first step can be referenced to determine that all of target tissue 158 has separated from muscular layer 160A.

In a third step of the ESD procedure, mucosal layer 160C can be cut to remove target tissue 158 from duct wall 160. In examples, medical instrument 168 can be used cut through mucosal layer 160C completely around the marked perimeter. Medical instrument 168 can include electrode ring 170 that can be activated by an energy source, such as at control unit 16 (FIG. 1), to cut through portions of mucosal layer 160C in order to facilitate separation of target tissue 158. Cutting of mucosal layer 160C can be performed so as to not penetrate muscular layer 160A. In other examples, mucosal layer 160C can be cut with a blade, forceps or the like.

In a fourth step of the ESD procedure, as shown in FIG. 5G, target tissue 158 can be removed. In examples, a medical instrument can be inserted through endoscope 104 to grab onto or attach to target tissue 158 including skirt 172 of mucosal layer 160C. Portions of submucosal layer 160B can remain attached to skirt 172 and muscular layer 160A (FIG. 5F) within cavity 174.

As mentioned, FIGS. 5E-5F describe one particular procedure in in which the disinfecting systems and methods can be used. In additional examples, the present disclosure can be used with other internal or endolumenal procedures like Polypectomy, Endoscopic Mucosal Resection (EMR), or Endoscopic Submucosal Dissection (ESD) that are not intended to puncture or otherwise penetrate through a duct wall. In additional examples, the present disclosure can be used with other internal or endoluminal procedures like Full-Thickness Resection (FTR) and endoscopic ultra-sound (EUS) drainage using a stent where penetration through an anatomic duct wall is desired and intended. The disinfectant or disinfecting of the present disclosure can be delivered before, after or during any of the steps described. In examples, the disinfectant can be delivered before the first two steps or before the cutting steps. In examples, disinfecting of the interior of duct 156 can be conducted with ultraviolet light, as described with reference to FIGS. 7A and 7B, or can be conducted with a coating on endoscope 104, as described with reference to FIG. 8.

FIG. 6A is perspective view of endoscopy system 200 comprising endoscope 204, medical instrument 206 and disinfectant instrument 208. FIG. 6B is a distal end view of endoscopy system 200 of FIG. 6A showing working channel 210, disinfectant channel 112, objective lens 214, illumination lens 216, and fluid outlet 218 in end face 220 of shaft 222. FIGS. 6A and 6B are discussed concurrently.

Medical instrument 206 can comprise a tissue retrieval device such as a forceps or any other device suitable for separating, retrieving or collecting sample biological matter. Medical instrument 206 can comprise shaft 230 and tissue separators 232. Shaft 230 of medical instrument 206 can comprise a pliable body that can allow tissue separator 232 to be angled out of elongate shaft 222. Shaft 230 can additionally accommodate passage of control features, such as actuation wires, to tissue separator 232 to facilitate actuation of tissue separator 232 to collect tissue.

Disinfectant instrument 208 can comprise shaft 240, spray device 242 and orifice 244. Disinfectant instrument 208 can be connected to 102 FIG. 5A similar to disinfectant instrument 108. Disinfectant can be emitted from spray device 242 to produce spray S.

Endoscopy system 200 of FIGS. 6A and 6B can be configured similarly as endoscopy system 100 of FIGS. 5A-5C except with disinfectant channel 112 omitted and disinfectant instrument 208 extending along the exterior of endoscope 204. Elements of endoscopy system 200 are provided with similar reference numbers with 100 series numbers replaced with 200 series numbers where appropriate. Disinfectant instrument 208 can be secured to the exterior of endoscope 204 using attachment device 260. As such, working channel 210 can be larger than if disinfectant instrument 208 were inside shaft 240 as can be seen in comparison to working channel 110 (FIG. 5C).

In examples, attachment device 260 can comprise a sleeve extending along shaft 222. In examples, attachment device 260 can comprise a hoop, strap or eyelet attached to shaft 222 and a plurality of attachment devices 260 can be positioned along the length of shaft 222. In additional examples, attachment device 260 can comprise a bulge in the exterior of shaft 22 that can incorporate disinfecting channel 112 (FIG. 5C) or a portion of the cross-sectional area of disinfecting channel 112. Likewise, though not shown in FIGS. 6A and 6B, shaft 222 can include a depression, channel or trough on the exterior surface into which disinfectant instrument 208 can be seated or partially seated to, for example, reduce the overall outer dimensions of endoscopy system 200. The depression, channel or trough can have a semi-circular or U-shaped cross-section to receive all or part of the thickness of 240. The depression, channel or trough can extend the length of shaft 230 or along a portion of shaft 230.

Positioning of disinfectant instrument 208 outside of shaft 222 can allow for the elimination of a disinfecting channel, such as disinfecting channel 112 of FIG. 5B, with one or both of the 1) reduction the cross-sectional area or diameter of shaft 222 and 2) increase of the cross-sectional area or diameter of working channel 210.

Spray device 242 can comprise a nozzle or another device for controlling the pattern of spray S by adjusting the size of orifice 244. For example, spray device 242 can be adjusted by an operator, such as at disinfecting system 102 (FIG. 2) or control unit 16 (FIGS. 1 and 2), to adjust the spray density, spray pattern, spray diameter, spray distance and the like. For example, spray device 242 can be adjusted to produce spray pattern S1 for dispensing disinfectant material in a concentrated manner over a longer distance or to produce spray pattern S2 for dispensing disinfectant material in a dispersed manner of a shorter distance. Spray device 242 can comprise various types of devices, such as a flat fan nozzle, a hollow cone nozzle, a full cone nozzle, a misting nozzle, a misting nozzle and an air atomizing nozzle. Spray device 242 can be connected to an actuator or motor that can be used to change the size of orifice 244.

FIG. 7A is perspective view of endoscopy system 300 comprising endoscope 304, medical instrument 306 and a light-based disinfectant system at disinfectant channel 370. FIG. 7B is a distal end view of endoscopy system 300 of FIG. 7A showing working channel 310, disinfectant channel 370, objective lens 314, illumination lens 316, fluid outlet 318 in end face 320 of shaft 322. FIGS. 7A and 7B are discussed concurrently.

Medical instrument 306 can comprise shaft 330 and tissue separators (not shown) and can be configured similarly to medical instrument 106 (FIGS. 5A and 5B) and medical instrument 206 (FIGS. 6A and 6B).

Endoscopy system 300 of FIGS. 7A and 7B can be configured similarly as endoscopy system 100 of FIGS. 5A-5C except with disinfectant channel 112 for providing a passageway for disinfectant substances or instruments being replaced by disinfectant channel 370 for receiving light conductor 372. Light conductor 372 can be connected to lens 374 at a distal end, linkage 376 and light ring 378. Elements of endoscopy system 300 are provided with similar reference numbers with 100 series numbers replaced with 300 series numbers where appropriate.

Light conductor 372 can be connected to light source 22 (FIG. 1) at a proximal end, either directly or through appropriate connectors. Light source 22 can be configured to generate light capable of disinfecting, such as ultraviolet light. In examples, light source 22 can generate UV-C short-wavelength ultraviolet radiation. UV-C wavelengths suitable for use with the present disclosure can be in a range of about 200 nanometers to about 300 nanometers. In other examples, other wavelengths or other types of light can be used.

Lens 374 can be positioned at the distal end of light conductor 372 to facilitate projecting of disinfecting light distally from endoscope 304. Lens 374 can be configured to disperse light in different angles from the end face of endoscope 304. Lens 374 can be configured as a light emitter configured to emit or project light carried by conductor 372 and originated by light source 22.

Light ring 378 can be connected to light conductor 372 via linkage 376. Light ring 378 can be configured to project light radially relative to the axis of endoscope 304. Linkage 376 can be made of a light conducting material. Light ring 378 can additionally be made of a light conducting material. Thus, light from light source 22 can be transmitted through light conductor 372, through linkage 376 and into light ring 378. In examples, light conductor 372, linkage 376 and light ring 378 can be made of one or more continuous light conductors, each connecting to one of emitters 379. Emitters 379 can be configured to emit disinfecting light radially from endoscope 304 relative to the central axis of endoscope 304. FIG. 7B illustrates four emitters 379 though more emitters 379 can be used to provide higher light density. In additional examples, emitters 379 can be located only along a circumferential segment of ring 378 (e.g., only within one one-hundred-eighty or ninety-degree segment) to provide disinfecting capabilities in a partial circumferential direction. Shaft 322 can be rotated to provide full circumferential disinfecting. Emitters 379 can be selectively activated by a user to provide disinfecting at a desired time. In examples, emitters 379 can be activated while shaft 322 is being delivered to an anatomic location to provide disinfecting leading up to target tissue. In example, emitters 379 can be activated as shaft 322 passes through an anatomic opening, either a natural opening between anatomic passageways, such as the esophagus, or an opening made during a procedure that passes through an anatomic duct, such as through a puncture in the stomach.

In additional examples, lens 374 can comprise a light emitter, such as one or more light emitting diodes (LEDs), and light conductor can comprise a wire or cable connecting lens 374 to a power source. Additionally, each of emitters 379 can comprise an LED or another light emitter configured to be electrically activated and linkage 376 and ring 378 can comprise wires or cables configured to conduct electrical power from light conductor 372 to emitters 379.

FIG. 8 is perspective view of endoscopy system 400 comprising endoscope 404, medical instrument 406 and disinfectant coating 480. Endoscope 404 can comprise working channel 410, objective lens 414, illumination lens 416, and fluid outlet 418 in end face 420 of shaft 422. FIGS. 7A and 7B are discussed concurrently.

Medical instrument 406 can comprise shaft 430 and tissue separators (not shown) and can be configured similarly to medical instrument 106 (FIGS. 5A and 5B) and medical instrument 206 (FIGS. 6A and 6B).

Endoscopy system 400 of FIG. 8 can be configured similarly as endoscopy system 100 of FIGS. 5A-5C except with disinfectant channel 112 being eliminated and disinfectant coating 480 being added. However, disinfectant coating 480 can be combined into any of the examples described herein, such as those described with reference to FIGS. 5A-7B. Elements of endoscopy system 400 are provided with similar reference numbers with 100 series numbers replaced with 400 series numbers where appropriate.

Disinfectant coating 480 can be fabricated from or include a disinfectant, such as those listed herein. The disinfectant can be embedded in or carried on the outside of disinfectant coating 480. Disinfectant coating 480 can be configured to rub off, abrade, elute, emit, molt, shed or otherwise release the disinfectant when sliding against or otherwise being in contact with another surface, such as an anatomic surface or biological tissue.

Disinfectant coating 480 can allow for passive disinfecting with endoscope 404 without direct intervention by the operator. Thus, coating 480 can provide disinfecting capabilities just by contacting tissue. In other configurations, disinfectant coating 480 can be configured to be released on demand by an operator with an activation source. In examples, shaft 422 can comprise resistance heaters that can be activated at control unit 16 (FIG. 1) to cause release of disinfectant from coating 480 via heating.

Disinfectant coating 480 can be located on all of shaft 422 or only on parts of shaft 422. For example, disinfectant coating 480 can be located only on the distal-most portion of shaft 422, such as the distal-most 50%, 25% or 10%. In additional examples, disinfectant coating 480 can be located only along a circumferential segment of shaft 422 (e.g., only within one one-hundred-eighty or ninety-degree segment) to provide disinfecting capabilities in a partial circumferential direction.

The including of disinfectant coating 480 can reduce the space used within shaft 422 for disinfecting purposes such that the space within shaft 422 can be available for other purposes. Thu, working channel 410 can be enlarged if desired, as compared to other working channels described herein.

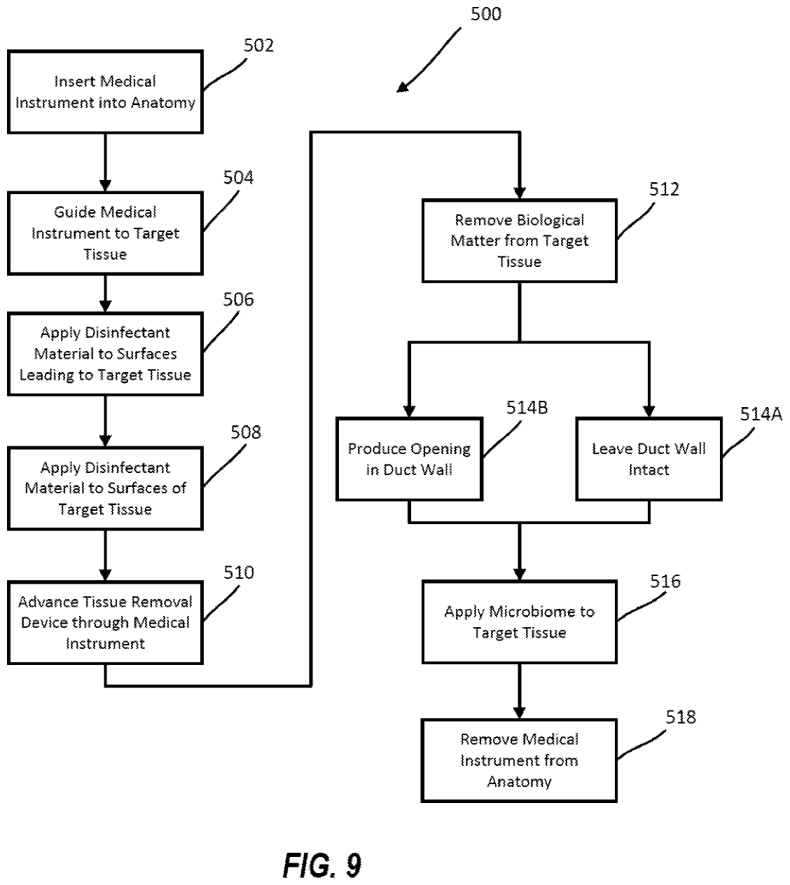

FIG. 9 is a line diagram illustrating methods for disinfecting surgical sites according to the present disclosure. Method 500 can comprise operation 502 through operation 518 that describe various procedures for disinfecting a surgical site of the present disclosure. In various examples, additional operations consistent with the systems, methods and operations described herein can be included, and some of operation 502-operation 518 can be omitted. Furthermore, operation 502-operation 518 can be performed in different orders than the illustrated example.

At operation 502, a medical instrument can be inserted into anatomy. For example, endoscope 104 can be inserted into duct D, as shown in FIG. 5A. In examples, an incision or opening can be made in the patient to reach the anatomy. In other examples, the medical instrument can be inserted into a body opening. In examples, endolumenal procedures can be performed within a duct of the anatomy.

At operation 504, the medical instrument can be guided to target tissue. For example, endoscope 104 can be guided to target tissue T, as shown in FIG. 5A. The target tissue can comprise diseased, potentially diseased or otherwise unwanted tissue, located inside of a patient. The target tissue can be tissue intended to be removed from the patient during the medical procedure or a location where an incision is to be made within tissue. In examples, the target tissue can comprise tissue located in various parts of the body, such as the urological tract, gynecological area, ear-nose-throat (ENT) area and stomach.

At operation 506, disinfectant can be applied by the medical instrument to surfaces leading to the target tissue. For example, disinfectant can be applied while the medical instrument is being inserted through the anatomy. In examples, the disinfectant can comprise ultraviolet light, as discussed with reference to FIGS. 7A and 7B. In examples, the disinfectant can comprise a coating applied to the exterior of the medical instrument, as discussed with reference to FIG. 8. In either case, the application of disinfectant can prevent the spread of bacteria and germs between different locations in the anatomy.

At operation 508, disinfectant can be applied to surfaces of the target tissue. For example, disinfectant instrument 108 can be advanced through working channel 112 to reach the target tissue, as shown in FIG. 5A. As discussed herein, disinfectant can be applied in a variety of ways, such as by being dispensed through the medical instrument, either using native fluid dispensing capabilities of the medical instrument or by use of an additional instrument inserted into the medical instrument, as shown in FIGS. 5A-5C, or attached to the outside of the medical instrument, as shown in FIGS. 6A and 6B. In examples, the disinfectant substance comprises Povidone-iodine (PVP-I) or Hypochlorous acid (HOCl).

At operation 510, a tissue removal device can be advanced through the medical instrument. For example, medical device or tissue separator device 106 of FIG. 5A can be advanced through working channel 110 to reach the target tissue.

At operation 512, biological matter can be removed from the target tissue. For example, tissue separator device 106 can be used to cut, slice or otherwise remove diseased or potentially diseased tissue or other biological matter. The tissue separator device can be removed from the anatomy to retrieve the biological matter for disposal or biopsy.

At operation 514A, the duct wall of the anatomy can be left intact. In examples, the medical procedure can comprise a Polypectomy, Endoscopic Mucosal Resection (EMR), or Endoscopic Submucosal Dissection (ESD) procedure. Disinfectant applied at operations 506 and 508 can prevent the spread of bacteria and germs onto the anatomy at the target tissue, thereby improving the health and safety of the patient by reducing the risk of infection or sickness.

At operation 514B, an opening in the duct wall of the anatomy can be made. In examples the opening can be made intentionally, such as during a full-thickness resection (FTR) or an endoscopic ultra-sound (EUS) drainage procedure. In other examples, the opening can be made unintentionally, such as during a procedure of operation 514A. In either case, the application of disinfectant at operations 506 and 508 can prevent the spread of bacteria and germs through the opening.

At operation 516, microbiome can be applied to the target tissue, as discussed in greater detail below. For example, bacteria of a patient can be supplemented or replaced with donor bacteria. The donor bacteria can supplement or replace bacteria killed via disinfectant applied at operation 506 and operation 508.

At operation 518, the medical instrument can be removed from the anatomy. For example, endoscope 104 can be removed from duct D, as shown in FIG. 5A. Any access incisions or openings made in the anatomy to facilitate access can be closed, such as via stitches, staples or sutures.

In another aspect of the present disclosure, the delivery devices and methods described herein can be used to perform microbiome procedures. In such procedures, the bacteria of a patient can be supplemented or replaced with donor bacteria, such as via a fecal transplant. Thus, it can be desirable to eliminate or remove the existing bacteria of the patient. The delivery devices and methods of the present disclosure can be used to deliver disinfectant to the anatomy to kill or remove the existing bacteria. The disinfectant can be delivered to a wider area than just a specific target tissue location. Thus, for example, the entire interior of the stomach can be treated with disinfectant by direct application. Thereafter, the delivery devices and methods of the present disclosure can be used to deliver donor bacteria, such as donor fecal matter, to the patient.

Any of the disinfectant systems of the present application can be used to deliver microbiomes to anatomy, particularly those configured to deliver powder or liquid disinfectant. Thus, for example, with reference to FIG. 5A, after disinfecting is performed with disinfectant instrument 108, disinfectant instrument 108 can be removed and another similar instrument configured to dispense microbiomes can be inserted into channel 112. Such an instrument can be fluidly coupled to a source of microbiomes at control unit 16 (FIG. 1). In examples, dispenser 142 can be used to deliver microbiomes, such as by providing fluid source 24 with microbiomes. In examples, a microbiome packet can be added to fluid source 24 at a desired point in time during a procedure to provide microbiomes. The microbiome packet can be manually added to fluid source 24 or can be added to fluid source 24 by an automatic dispensing mechanism of control unit 16 (FIG. 1). In additional examples, disinfectant instrument 108 can be used to dispense disinfectant and microbiomes, such as by switching the source of material disinfectant instrument 108 is connected to at a proximal end portion.

### EXAMPLES

Example 1 is a method of performing a surgical procedure comprising: inserting a medical instrument into anatomy of a patient to reach target tissue within an anatomical cavity; disinfecting anatomy within the patient with the medical instrument; and performing treatment of the target tissue with the medical instrument.

In Example 2, the subject matter of Example 1 optionally includes wherein performing treatment of the target tissue comprises removing the target tissue from a duct wall of the anatomical cavity using a tissue removal device inserted through the medical instrument.

In Example 3, the subject matter of Example 2 optionally includes wherein removing the target tissue from the duct wall of the anatomical cavity comprises leaving the duct wall intact.

In Example 4, the subject matter of any one or more of Examples 2-3 optionally include wherein the surgical procedure comprises one of: Polypectomy, Endoscopic Mucosal Resection (EMR), or Endoscopic Submucosal Dissection (ESD).

In Example 5, the subject matter of any one or more of Examples 2-4 optionally include wherein: the surgical procedure further comprises producing an opening in the duct wall; and disinfecting anatomy within the patient with the medical instrument comprises applying disinfectant around the opening.

In Example 6, the subject matter of any one or more of Examples 2-5 optionally include wherein the surgical procedure comprises a full-thickness resection (FTR) or an endoscopic ultra-sound (EUS) drainage.

In Example 7, the subject matter of any one or more of Examples 1-6 optionally include wherein disinfecting the anatomy comprises dispensing disinfectant material onto tissue surrounding the target tissue through the medical instrument.

In Example 8, the subject matter of Example 7 optionally includes wherein dispensing disinfectant material onto the anatomy through the medical instrument comprises dispensing the disinfectant material from a tubular body inserted into the medical instrument.

In Example 9, the subject matter of any one or more of Examples 7-8 optionally include wherein dispensing the disinfectant material onto the target tissue comprises using native fluid dispensing capabilities of the medical instrument to dispense the disinfectant material.

In Example 10, the subject matter of Example 9 optionally includes adding a disinfectant packet to a fluid reservoir for the native fluid dispensing capabilities of the medical instrument.

In Example 11, the subject matter of any one or more of Examples 7-10 optionally include wherein dispensing the disinfectant material onto the target tissue comprises: using a first fluid dispensing capability of the medical instrument to dispense irrigation fluid; and using a second fluid dispensing capability of the medical instrument do dispense the disinfectant material.

In Example 12, the subject matter of any one or more of Examples 7-11 optionally include wherein dispensing disinfectant material onto the anatomy through the medical instrument comprises dispensing the disinfectant material from a tubular body attached to an exterior of the medical instrument.

In Example 13, the subject matter of any one or more of Examples 7-12 optionally include wherein dispensing the disinfectant material onto the target tissue comprises dispensing Povidone-iodine (PVP-I) or Hypochlorous acid (HOCl).

In Example 14, the subject matter of any one or more of Examples 7-13 optionally include adjusting a surface area over which the disinfectant material is distributed onto the target tissue.

In Example 15, the subject matter of any one or more of Examples 7-14 optionally include wherein dispensing disinfectant material onto tissue surrounding the target tissue comprises applying cold plasma to the target tissue.

In Example 16, the subject matter of any one or more of Examples 7-15 optionally include wherein dispensing disinfectant material onto tissue surrounding the target tissue comprises applying ultraviolet light to the target tissue. In Example 17, the subject matter of any one or more of Examples 1-16 optionally include wherein disinfecting the anatomy comprises dispensing disinfectant material onto tissue leading up to the target tissue.

In Example 18, the subject matter of Example 17 optionally includes wherein disinfecting the anatomy comprises directing ultraviolet light radially outward from the medical instrument.

In Example 19, the subject matter of any one or more of Examples 17-18 optionally include wherein disinfecting the anatomy comprises rubbing a disinfecting coating applied to the medical instrument onto the anatomy.

In Example 20, the subject matter of any one or more of Examples 1-19 optionally include dispensing a microbiome onto the anatomy.

Example 21 is a medical device for providing disinfecting to a surgical site, the medical device comprising: an elongate shaft extending from a proximal end to a distal end; an imaging device located proximate the distal end of the elongate shaft; a lighting element located proximate the distal end of the elongate shaft; a working channel extending at least partially through the elongate shaft; and a disinfecting system attached to the elongate shaft to deliver a disinfecting capability proximate the distal end of the elongate shaft.

In Example 22, the subject matter of Example 21 optionally includes wherein: the disinfecting system comprises a disinfectant tube; and the disinfecting capability comprises dispensing of a disinfecting substance.

In Example 23, the subject matter of Example 22 optionally includes wherein the disinfectant tube extends within the elongate shaft.

In Example 24, the subject matter of any one or more of Examples 22-23 optionally include wherein the disinfectant tube extends alongside the elongate shaft.

In Example 25, the subject matter of Example 24 optionally includes an attachment device to hold the disinfectant tube alongside the elongate shaft.

In Example 26, the subject matter of any one or more of Examples 22-25 optionally include an adjustable nozzle connected to the disinfectant tube to change a spray diameter from the disinfectant tube.

In Example 27, the subject matter of any one or more of Examples 22-26 optionally include wherein the disinfecting substance comprises a liquid or powder comprising Povidone-iodine (PVP-I) or Hypochlorous acid (HOCl).

In Example 28, the subject matter of any one or more of Examples 22-27 optionally include wherein the disinfecting substance comprises a gas.

In Example 29, the subject matter of Example 28 optionally includes wherein the gas comprises cold plasma.

In Example 30, the subject matter of Example 29 optionally includes wherein the disinfecting capability comprises: a conductor extending along the disinfectant tube; and an electrode connected to a distal end portion of the conductor.

In Example 31, the subject matter of any one or more of Examples 21-30 optionally include wherein: the disinfecting system comprises a conductor; and the disinfecting capability comprises disinfecting light capable of being emitted from the medical device.

In Example 32, the subject matter of Example 31 optionally includes wherein the disinfecting light is emitted radially.

In Example 33, the subject matter of any one or more of Examples 31-32 optionally include wherein the disinfecting light is emitted axially.

In Example 34, the subject matter of any one or more of Examples 31-33 optionally include wherein the conductor comprises a light fiber and the disinfecting system further comprises a light-emitting diode.

In Example 35, the subject matter of any one or more of Examples 31-34 optionally include the disinfecting light comprises ultraviolet having a wavelength in a range of approximately 200 nm to approximately 280 nm.

In Example 36, the subject matter of any one or more of Examples 22-35 optionally include wherein: the disinfecting system comprises a coating positioned on at least a portion of an exterior of the elongate shaft; and the disinfecting capability comprises a disinfectant substance within the coating.

In Example 37, the subject matter of Example 36 optionally includes wherein the disinfectant substance comprises Povidone-iodine (PVP-I) or Hypochlorous acid (HOCl).

In Example 38, the subject matter of any one or more of Examples 22-37 optionally include an irrigation passage extending through the elongate shaft.

In Example 39, the subject matter of Example 38 optionally includes wherein: the disinfecting system comprises the irrigation passage; and the disinfecting capability comprises irrigation fluid having a disinfectant additive.

In Example 40, the subject matter of Example 39 optionally includes packets of disinfectant.

Each of these non-limiting examples can stand on its own, or can be combined in various permutations or combinations with one or more of the other examples.

### Various Notes

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventor also contemplates examples in which only those elements shown or described are provided. Moreover, the present inventor also contemplates examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A method of performing a surgical procedure comprising:
inserting a medical instrument into anatomy of a patient to reach target tissue within an anatomical cavity;
disinfecting anatomy within the patient with the medical instrument; and
performing treatment of the target tissue with the medical instrument.

2. The method of claim 1, wherein performing treatment of the target tissue comprises removing the target tissue from a duct wall of the anatomical cavity using a tissue removal device inserted through the medical instrument.

3. The method of claim 2, wherein removing the target tissue from the duct wall of the anatomical cavity comprises leaving the duct wall intact.

4. The method of claim 2, wherein the surgical procedure comprises one of: Polypectomy, Endoscopic Mucosal Resection (EMR), or Endoscopic Submucosal Dissection (ESD).

5. The method of claim 2, wherein:
the surgical procedure further comprises producing an opening in the duct wall; and
disinfecting anatomy within the patient with the medical instrument comprises applying disinfectant around the opening.

6. The method of claim 2, wherein the surgical procedure comprises a full-thickness resection (FTR) or an endoscopic ultra-sound (EUS) drainage.

7. The method of claim 1, wherein disinfecting the anatomy comprises dispensing disinfectant material onto tissue surrounding the target tissue through the medical instrument.

8. The method of claim 7, wherein dispensing disinfectant material onto the anatomy through the medical instrument comprises dispensing the disinfectant material from a tubular body inserted into the medical instrument.

9. The method of claim 7, wherein dispensing the disinfectant material onto the target tissue comprises using native fluid dispensing capabilities of the medical instrument to dispense the disinfectant material.

10. The method of claim 9, further comprising adding a disinfectant packet to a fluid reservoir for the native fluid dispensing capabilities of the medical instrument.

11. The method of claim 7, wherein dispensing the disinfectant material onto the target tissue comprises:
using a first fluid dispensing capability of the medical instrument to dispense irrigation fluid; and
using a second fluid dispensing capability of the medical instrument do dispense the disinfectant material.

12. The method of claim 7, wherein dispensing disinfectant material onto the anatomy through the medical instrument comprises dispensing the disinfectant material from a tubular body attached to an exterior of the medical instrument.

13. The method of claim 7, wherein dispensing the disinfectant material onto the target tissue comprises dispensing Povidone-iodine (PVP-I) or Hypochlorous acid (HOCl).

14. The method of claim 7, further comprising adjusting a surface area over which the disinfectant material is distributed onto the target tissue.

15. The method of claim 7, wherein dispensing disinfectant material onto tissue surrounding the target tissue comprises applying cold plasma to the target tissue.

16. The method of claim 7, wherein dispensing disinfectant material onto tissue surrounding the target tissue comprises applying ultraviolet light to the target tissue.

17. The method of claim 1, wherein disinfecting the anatomy comprises dispensing disinfectant material onto tissue leading up to the target tissue.

18. The method of claim 17, wherein disinfecting the anatomy comprises directing ultraviolet light radially outward from the medical instrument.

19. The method of claim 17, wherein disinfecting the anatomy comprises rubbing a disinfecting coating applied to the medical instrument onto the anatomy.

20. The method of claim 1, further comprising dispensing a microbiome onto the anatomy.
